(19) United States
(12) Patent Application Publication
(10) Pub. No.: US 2023/0238105 A1
(43) Pub. Date: Jul. 27, 2023

(54) DRUG DELIVERY DEVICE WITH INCREASED SAFETY

(71) Applicant: Ypsomed AG, Burgdorf (CH)

(72) Inventors: Andreas Schneider, Basel (CH); Leos Urbanek, Bern (CH); Reto Jost, Grosshöchstetten (CH); Patrick Hostettler, Hasle (CH)

(21) Appl. No.: 18/295,386

(22) Filed: Apr. 4, 2023

Related U.S. Application Data
(63) Continuation of application No. PCT/EP2021/079196, filed on Oct. 21, 2021.

(30) Foreign Application Priority Data
Oct. 29, 2020 (EP) 20204580.3

(51) Int. Cl. G16H 20/17; A61M 5/142; A61M 5/20; A61M 5/315

(52) U.S. Cl. CPC G16H 20/17; A61M 5/14248; A61M 5/20; A61M 5/31591; A61M 2205/0266; A61M 5/19

(57) ABSTRACT

A disposable, single-dose delivery device for self-administration by a patient of a predefined amount or dose of drug from a reservoir through an outlet of the reservoir includes a device operation lock to prevent a delivery operation of the delivery device as shipped to the patient, an embedded electronic control unit with a wireless receiver for receiving an unlock command or message, and an electromechanical actuator for mechanically unlocking the device operation lock and enabling delivery device operation by the patient. The control unit is configured to activate the actuator instantaneously and unconditionally upon receipt of the unlock command. The unlocking mechanism of the delivery device is adapted to be activated from remote in response to a confirmative message indicative of additional double check by a Health Care Professional HCP or expert system.

### CROSS REFERENCE TO RELATED APPLICATIONS

This application claims priority to International Patent Application No. PCT/EP2021/079196, filed Oct. 21, 2021, entitled “DRUG DELIVERY DEVICE WITH INCREASED SAFETY,” which in turn claims priority to European Patent Application No. 20204580.3, filed Oct. 29, 2020, entitled “DRUG DELIVERY DEVICE WITH INCREASED SAFETY”, each of which is incorporated by reference herein, in the entirety and for all purposes.

### TECHNICAL FIELD

The present invention relates to medicament delivery devices for delivering, administering, dispensing, injecting, or infusing substances and/or liquids such as insulin or hormone preparations. It departs from a delivery device for self-administration of a predefined amount of drug from a reservoir.

### BACKGROUND

A variety of diseases exist that require regular treatment by parenteral subcutaneous or intramuscular administration of a drug or medicament, and a number of drug delivery devices have been developed to support a patient in accurately and controllably delivering an amount of drug in a self-administration process. Delivery devices include injection devices that are removed from the injection site after each medication event or drug delivery process. An injection pen device has an elongate device body defining a longitudinal main device axis. The term “distal end” refers to the end of the injection device where an injection needle or cannula is located, the term “proximal end” designates the opposite end thereof. Delivery devices further include infusion devices or pumping devices for continual delivery of a regular or basal amount of medication through a fluid channel into the body of the patient, wherein the fluid channel remains in use for a prolonged period of time.

Disposable or single-use delivery devices are adapted to deliver a drug from a container such as a pre-filled syringe that is not intended to be replaced or refilled by the patient. Reusable, semi-reusable, or hybrid delivery devices have a container that may be replaced by the patient, or a cartridge that may be refilled, while some components of the device may be reused with the replaced or refilled drug container. An automatic injection device has an electric motor or a drive spring for biasing a piston rod and shifting a piston in a container barrel, wherein the drive spring may have to be charged or strained manually prior to injection of a dose. A manually powered injection device requires a user to manually provide the energy to move the piston, for instance by applying a distal force component to the injection device.

Drug delivery device based therapies generally benefit from an electronic unit or control unit embedded or integrated in the delivery device. The electronic unit monitors a drug delivery process, in order to proactively prevent false handling of the device and/or to keep track of the doses already applied, and generates data related to an instantaneous condition and/or use of the delivery device. Suitable sensors of the electronic unit readily detect a status or signal from any kind of indicating component of the delivery device, including user interface elements and actuators. A wireless communication unit of the electronic unit is provided to wirelessly communicate, specifically upload, drug delivery information to a nearby mobile device or dedicated medical gateway. The drug delivery information includes at least a time stamp and the expelled dose, indicative of a time of a medication event and of a quantity of delivered medicament. The drug delivery information may be transmitted instantaneously, or stored in a memory unit connected to the processing unit, for later upload or batch transfer.

EP 3013390 B1 discloses a bolus-only infusion pump that enables delivery of insulin via a mechanical drive mechanism that is controlled by the patient. In order to deliver a bolus the patient has to enter the amount into a remote controller. Subsequently, the controller sends a message to the pump that actuates a motor to unlock the mechanical drive mechanism. The patient dials the number of clicks at the pump to deliver medication. Once the desired amount is delivered a locking mechanism automatically engages disabling further delivery of medication.

EP 2879735 B1 discloses a cassette unit for an electrically powered auto-injector with a re-useable drive unit. The cassette unit is adapted to prevent accidental or unintended removal of a removable front-cap from the cassette unit by means of a shuttle lock control movable from a first ‘cassette unused’ position to a second ‘cassette unlocked’ position, in which it no longer prevents such cap removal. The shuttle lock control is configured to be manually releasable or automatically releasable by interaction with a suitable feature of the drive unit when the cassette unit locates at the docking position within the drive unit housing.

U.S. Pat. No. 8,882,722 B2 discloses a reusable injection pen with a protective cap that is releasably retained over a distal end of a cartridge housing. A lockout feature is positioned between the cap and a dose setting mechanism and is configured to time lock the cap to the dose setting mechanism to prevent an authorized injection. The lockout feature is settable wirelessly from a remote location, to open or close at a specific time of day, or day of the week, or week of a month, according to a schedule pre-programmed by a Health Care Professional. After an injection has been made, the user can replace the replaceable cap to re-cover the cartridge housing. Once replaced a time lock will lock the cap to the dose setting mechanism until it is time for the next injection.

In the present context, the terms “substance”, “drug”, “medicament” and “medication” are to be understood to include any flowable medical formulation suitable for controlled administration through a means such as, for example, a cannula or a hollow needle, and comprises a liquid, a solution, a gel or a fine suspension containing one or more medical active ingredients. A medicament can be a composition comprising a single active ingredient or a pre-mixed or co-formulated composition with more than one active ingredient present in a single container. Medication includes drugs such as peptides (e.g., insulin, insulin-containing drugs, GLP-1 containing drugs or derived or analogous preparations), proteins and hormones, active ingredients derived from, or harvested by, biological sources, active ingredients based on hormones or genes, nutritional formulations, enzymes and other substances in both solid (suspended) or liquid form but also polysaccharides, vaccines, DNA, RNA, oligonucleotides, antibodies or parts of antibodies but also appropriate basic, auxiliary and carrier substances.

### SUMMARY

It is an objective of the invention to increase therapeutic safety and patient comfort in self-administration of drugs by means of a drug delivery device operated by a patient, while maintaining a certain level of flexibility. This objective is achieved by a device and a method according to the independent claims. Preferred embodiments are evident from the dependent patent claims.

According to the invention, a disposable, single-dose delivery device for self-administration, by a patient or user, of a predefined amount or dose of drug from a reservoir through an outlet of the reservoir, is adapted to prevent or inhibit a delivery operation of the delivery device as shipped to the patient. The delivery device has a device operation lock, an embedded electronic control unit with a wireless receiver for receiving an unlock command or message, and an actuator for unlocking, in particular an electromechanical actuator for mechanically unlocking, the device operation lock and enabling delivery device operation by the patient. The control unit is configured to activate the actuator instantaneously and unconditionally upon receipt of the unlock command. The unlocking mechanism of the delivery device is adapted to be activated from remote in response to a confirmative message indicative of a supplemental or double check by a Health Care Professional HCP or expert system. Receipt of the unlocking message is a necessary condition for a delivery operation, and introduces an additional layer of therapeutic safety and patient comfort. Unlocking of the device operation lock commences instantaneously and without delay, and does not depend on pre-defined schedules or criteria applied to actual environmental conditions at the place of the patient.

The delivery device may be an injection device of the kind described in international patent application publication WO 2021/160540 A1, and in particular comprise some or all of the components described therein, and/or be assembled from some or all of these components. Said components may in particular comprise: one or more of a syringe holder; a plunger comprising a plunger rod (“Vortriebsglied”) for displacing a drug contained in the reservoir through an injection needle; a spring, in particular a dispense spring (“Ausschüttfeder”) for actuating the plunger rod and/or the plunger; a removable device (front-)cap, also referred to as a cap remover (“Abziehkappe”); and a cover sleeve (“Nadelschutzhülse”) for covering the needle prior to and/or after the injection to avoid injury, including unintentional skin penetration, to the subject or other persons. The delivery device may be an electrically powered auto-injector, for which the device operation lock of a mechanical dispense trigger such as a push button or trigger sleeve may supplement an electronic control of the dispense motor.

In an initial, active, activated and/or locked state, the device operation lock may at least partially inhibit and/or prevent an operation, in particular the delivery operation, of the delivery device. To this end, the device operation lock in its initial, active, activated and/or locking state may inhibit and/or prevent movement, displacement, rotation, deformation and/or operation of one or more components of the delivery device, in particular of the cap remover, the cover sleeve, the syringe holder, the dispense spring, the plunger and/or the plunger rod, and may thus effectively lock the delivery device. This may, in particular, be achieved by the device operation lock, or at least a part thereof, engaging one or more of the components of the delivery device, in particular to establish a form lock or positive locking fit. If an attempt is made by the patient or another user of the delivery device to initiate, start and/or execute the delivery operation, the patient or the other user may exert a force, in particular a tensile force, a compressive force and/or a shearing force, onto one or more components of the delivery device, or may activate and/or release mechanical energy stored in a mechanical energy storage or mechanical energy storage element, in particular a (pre-)biased elastic element, e.g. a spring, to exert such force, in order to effect movement, displacement, rotation, deformation and/or operation of said one or more components. The force may be exerted directly or indirectly, i.e. through and/or via a further component. If the device operation lock is in its initial, active, activated and/or locking state when such attempt is made, it may absorb and thus counteract and/or neutralize the force, and may thus block movement, displacement, rotation, deformation and/or operation of said one or more components. As a consequence, movement, displacement, rotation, deformation and/or operation of said one or more components will be inhibited and/or prevented. The device operation lock may be configured and/or designed such that it can withstand the force without being deformed, broken, shattered, elongated, ripped, torn or otherwise altered in a manner in which it will no longer inhibit and/or prevent operation, in particular delivery operation, of the delivery device. In particular, the device operation lock may be designed such that it will not be deformed in a manner that would alter its shape beyond, or at least not significantly beyond, what is permitted by applicable manufacturing tolerances when subjected to a maximum expectable force. The maximum expectable force may be defined by a characteristic, in particular a Young's modulus or a modulus of elasticity, of the (pre-) biased elastic element, in particular the spring, and an amount of (pre-) bias applied, or by a magnitude of force the patient or other user may be expected to exert when intending to use the delivery device, which may in particular be 50N or 100N, and/or may be determined by experiment and/or statistics.

More specifically, when the device operation lock in its initial, active, activated and/or locking state inhibits and/or prevents movement or displacement, in particular removal, of the cap remover, it may be ensured that a distal end region of the delivery device, in particular of the injection needle or cannula, remains sterile as long as no unlock command or message has been received. When the device operation lock in its initial, active, activated and/or locking state inhibits and/or prevents movement or displacement of the cover sleeve and/or the syringe holder, it may be ensured that the patient's skin may not be penetrated as long as no unlock command or message has been received. When the device operation lock in its initial, active, activated and/or locking state inhibits and/or prevents movement or displacement of the plunger, the plunger rod and/or the dispense spring, it may be ensured that no drug or medicament may be dispensed as long as no unlock command or message has been received. In a preferred embodiment, the delivery device is an auto-injector or a patch-injector, with a drive means such as a preloaded spring, for automated delivery, upon operation of a patient-operable trigger element, of a predefined, non-user selectable amount of drug. The amount of drug delivered is preferably the entire volume of the reservoir, but in exceptional circumstances, the device might be limited to deliver a predefined fraction, with the rest being discarded after use. The auto-injector has a needle cover sleeve as a trigger element and, preferably, no needle or syringe movement, while the patch-injector has a cannula insertion mechanism activated by a trigger button. The invention may also be beneficially applied to a multi-variable-dose injection device for repeated delivery of variable doses of drug from a container that may or may not be intended to be replaced by the patient. In this case the device locking mechanism is adapted for multiple operations, i.e. capable of re-locking at the end of a dose delivery operation.

In a preferred embodiment, the device operation lock includes an outlet cover lock preventing removal of a non-replaceable outlet cover sealing, and maintaining sterile and uncontaminated, the outlet of the reservoir. Specifically, the outlet lock is a device cap lock of the auto-injector preventing removal of a device cap and/or needle shield of the auto-injector, or a barrier lock of the patch injector preventing removal of a sterile barrier and/or release liner of the patch-injector. The device operation lock thus prevents the outlet from being exposed to ambient in the first place, such that the delivery device remains sterile and may be used at a later time. In a less preferred embodiment, the outlet cover is adapted to be removed and the device operation lock is adapted to lock the drive mechanism or trigger element instead, such that a locked device would have to be discarded in case the user inadvertently removes the outlet cover in the first place.

In a refined variant the electronic control unit includes a timer and is configured to control the electromechanical actuator to mechanically re-lock or activate the device operation lock after lapse of a certain unlock delay or interval. Operation of the sterile and safe-to-use device is then disabled until a next unlock command is received. Re-locking may also take place in other cases, for instance if and/or as long as wireless communication to or from the delivery device is interrupted.

In a preferred embodiment, the device cap lock includes a flexible ledge engaging, in a locked or shipping state, a rigid counter-ledge, and prevented by a locking slider from disengaging the counter-ledge. One of the ledges is part of the device cap while the other is part of the device housing. The locking slider conveys an unlocking movement of the actuator to or towards the device operation lock, such that the actuator may be located at a distance from the latter, specifically in a proximal or rear part of the device. The unlocking movement of the locking slider may include an axial hub of 1 to 5 mm along a main axis of the delivery device.

In a preferred embodiment the electronic control unit includes a wireless transmitter to send an unlock request message to the HCP or expert system, triggered by a user input or activity detected by the delivery device. The HCP or expert system may respond automatically with the unlock command if and as soon as the necessary conditions or requirements are deemed to be satisfied by the HCP or expert system. The unlock request message may also help to establish that a communication link to a therapy management system is in fact active and available for transmission of the expected unlock command.

In a refined variant the electronic control unit includes a device-activation sensor operative at no or lowest power consumption during storage and shipping of the delivery device, and adapted to detect a user-initiated preparation of the delivery device indicative of a forthcoming operation of the device. The device-activation sensor means may include a touch sensor, a motion sensor, a temperature sensor, a tamper sensor, or an electromechanical switch coupled to and activated by a device packaging lid or cover. The device-activation sensor means may detect a tentative device cap removal action by the patient, including a push, pull, or twist movement with small amplitude of the locked device cap with respect to the device housing, in order to activate the electronic control unit, a display, and/or the unlock request process. The device-activation sensor means may detect tentative or partial removal of an adhesive release liner or sterile barrier of a patch-injector. The electronic control unit is configured to activate or wake-up from an idle or stand-by state based on a corresponding response of the device-activation sensor, and initiate the unlock request message transmission.

In a preferred embodiment the electronic control unit of the delivery device includes a drug delivery sensor such as the rotation sensor disclosed in the patent application CH 715791 A2 to monitor a drug delivery operation of the device. The electronic unit is further adapted to produce a time-stamp indicating at what date and time a monitored dose has been dispensed, or to start a counter indicative of how long ago the dose dispense has occurred. The electronic unit is adapted to store delivery data including the dose delivered, the time-stamp, and a quality of the delivery including holding time in a data storage unit, and to upload the latter and/or a counter value at upload time to the HCP or other stakeholder. The electronic unit of the delivery device may further include a device status indicator that provides visual feedback about a device or process status including an availability of battery power, a readiness of communication means, or a progress of an ongoing delivery process.

In a preferred embodiment, the actuator includes a shape-memory-alloy element in the form of a wire, which is capable of producing the required hub without additional mechanical gearing. Preferably, the shape memory alloy wire has a diameter of 0.01-1 mm, specifically 0.05-0.1 mm, and/or a length of 30-100 mm, specifically 50-70 mm, and may develop a hub of 1-5 mm, specifically 1.5-3 mm within a few seconds when heated to a critical temperature. The wire dimensions in the range indicated allow a reasonably sized energy source or battery to provide the necessary heating power.

In further preferred embodiments of the delivery device, the device operation lock may consist of or comprise a thermosensitive element, which may melt, evaporate, dissolve, soften and/or otherwise become structurally weakened when subjected to an elevated temperature Tᵣₑₗₑₐₛₑ, in particular a temperature Tᵣₑₗₑₐₛₑ above, in particular substantially larger, than a temperature of the human body or a temperature of a hot, in particular tropical, natural environment. This may, in particular, be achieved by forming the thermosensitive element from a thermosensitive material, as, e.g., described in European patent application EP 3 501 577 A1, where reference is made to a softenable or meltable material (“aus einem aufweichbaren oder schmelzbaren Material”, cf. paragraph 0063). Exemplary values for Tᵣₑₗₑₐₛₑ may be 60° C., preferably 70° C., most preferably 85° C.

The device operation lock and/or the thermosensitive element may be configured such that when the device operation lock prevents and/or inhibits movement, displacement, rotation, deformation and/or operation of one or more components of the delivery device by absorbing—and thus counteracting and/or neutralizing—a force exerted onto the one or more components of the delivery device as described further above, said force will at least partially act onto the thermosensitive element. This may, in particular, be achieved by forming the thermosensitive element in a manner, in particular from a material, as described in connection with the meltable Element (“erweichbare[s] oder schmelzbare[s] Element”, “Schmelzelement”, “Schmelz-sicherungselement”, “Schmelzsicherung”, etc) of EP 3 501 577 A1, in particular paragraphs 0098 through 0138. The material may, in particular, be metallic or plastic, in particular polymeric, as e.g. acrylonitrile butadiene styrene (ABS). Plastic material may be either electrically non-conducting or electrically conducting, wherein conductivity may be either intrinsic or due to fillers.

Unlocking of the device operation lock consisting of or comprising the thermosensitive element may be achieved by heating said thermosensitive element to a temperature corresponding to or exceeding Tᵣₑₗₑₐₛₑ, which may transition the device operation lock from its initial, active, activated and/or locked state to an inactive, deactivated and/or unlocked state. In the deactivated and/or unlocked state, the device operation lock, in particular the thermosensitive element, in as far as it has not melted away, dissolved or evaporated, will no longer be able to withstand, in particular absorb, counteract and/or neutralize, the force exerted on it due to the attempt the patient or another user to initiate, start and/or execute the delivery operation of the delivery device, but will be deformed, broken, shattered, elongated, ripped, torn or otherwise altered in a manner such that it will no longer block, prevent and/or inhibit movement, displacement, rotation, deformation and/or operation of the involved one or more components.

When the thermosensitive element is at least essentially made of plastic material, the elevated temperature Tᵣₑₗₑₐₛₑ may be chosen to correspond to a glass transition temperature of said material, or to a value significantly larger than said glass transition temperature. Alternatively, the elevated temperature Tᵣₑₗₑₐₛₑ may be chosen to correspond to a heat deflection temperature or heat distortion temperature (HDT, HDTUL, or DTUL) of the plastic material, or to a value significantly larger than said heat deflection temperature or heat distortion temperature. When the thermosensitive element is at least essentially made of metal, in particular Sn, Pb and/or Zn, the elevated temperature Tᵣₑₗₑₐₛₑ may be chosen to correspond to a melting temperature of said material, or to a value significantly larger than said melting temperature.

When the thermosensitive element comprises electrically conducting material, in particular metallic material or electrically conducting plastic material, heating of said thermosensitive element to a temperature corresponding to or exceeding Tᵣₑₗₑₐₛₑ may be achieved by electrically connecting the thermosensitive element to an electric power source, in particular to a voltage source or to a current source to cause an electric current to flow through the electrically conducting material, which may then heat up due to electric losses in said material, an particular due to an electric resistivity of the material.

When the thermosensitive element is non-conducting and/or electrically insulating, a separate electric heating element may need to be provided in close proximity to the device operation lock and/or thermosensitive element to supply an amount of heat and/or thermal energy required to melt, evaporate, dissolve, soften and/or otherwise structurally weaken the thermosensitive element. In particular, the stretch and/or one or more loops of conductor comprised by the heating element may be attached to, pressed against, or wrapped around the device operation lock, in particular the thermosensitive element, or otherwise be provided in physical contact, in particular direct physical contact, with the latter.

In embodiments in which the device operation lock comprises a thermosensitive element as described above, the actuator may comprise heating means which allow to heat the device operation lock and/or the thermosensitive element, in particular to a temperature above Tᵣₑₗₑₐₛₑ. The heating means may comprise circuitry, in particular electric and/or electronic circuitry, which may in turn comprise an electric heating element, which heating element may in particular comprise a stretch and/or one or more loops of conductor, in particular of resistive wire. The circuitry may allow to selectively connect the thermosensitive element and/or the heating element to an electric power source, in particular a voltage or current source, in order to effect an electric current flow through thermosensitive element and/or the heating element, respectively. The circuitry may comprise switching elements and/or switches which may allow for an electric connection of the thermosensitive element and/or the heating element to the electric power source to be established or separated, in particular under control of the control unit. The control unit may be configured to, upon receipt of the unlock command, connect the thermosensitive element and/or the heating element to the electric power source, in particular for a predetermined length of time after which it may separate the thermosensitive element and/or the heating element from the electric power source. Alternatively and/or additionally, the temperature of the thermosensitive element may be monitored by a temperature sensor, which may provide feed-back to the control unit, which may then disconnect the electric power source as soon as the thermosensitive element has been heated sufficiently.

The electric energy source may be part of the actuator, and may in particular be a dedicated electric power source exclusively provided for the purpose of heating the device operation lock and/or the thermosensitive element upon receipt of the unlock command. Alternatively, the power or voltage source may be part of the delivery device, and may be configured to provide electric power to other components and/or functions of said delivery device, in particular to the wireless receiver and/or the control unit. The electric energy source may, in particular, be a (rechargeable) battery or a pre-charged capacitor.

An amount of heat and/or thermal energy supplied to the thermosensitive element may be (pre-) determined and/or controlled through the current and/or voltage applied to the electrically conducting material or the heating element, and a period of time during which said current and/or voltage is applied, and will further depend on a resistivity of the electrically conducting material or the heating element, in particular a resistivity of the stretch and/or the one or more loops of conductor.

The temperature to which thermosensitive element will be heated may be controlled by (pre-)determining and or controlling the amount of heat and/or thermal energy supplied to the thermosensitive element as described above by further taking into account, inter alia, a heat capacity of the thermosensitive element, and possibly the device operation lock. This may allow to control a degree to which the thermosensitive element will melt, evaporate, dissolve, soften and/or otherwise be structurally weakened. In particular, the amount of heat and/or thermal energy may be chosen such that the thermosensitive element will not melt, evaporate, dissolve or otherwise become substantially, in particular irreversibly, structurally weakened, but will only soften and/or become deformable. If, in such a situation, no attempt is made by the patient or another user of the delivery device to initiate, start and/or execute the delivery operation within a certain amount of time, in particular within 1 minute, 5 minutes or 10 minutes, the heat and/or thermal energy previously supplied to the thermosensitive element may dissipate, which may in particular lead to the thermosensitive element to cool down to a temperature below Tᵣₑₗₑₐₛₑ. This may in turn revert the softening and revert the thermosensitive element to an at least essentially non-deformable, rigid state, thus effectively relocking the delivery device, and/or conditionally upon receipt of the unlock command.

The heating element may comprise a thermoelectric element, in particular a thermocouple, provided in close proximity to, in particular direct physical contact with, the device operation lock and/or the thermosensitive element, and may allow to heat the thermosensitive element through application of the Peltier effect. Such a thermoelectric element may alternatively be used to actively cool the device operation lock and/or the thermosensitive element, in particular by reverting a polarity of the connection to the electric power source as compared to a heating operation, and may accelerate relocking the delivery device, and/or render relocking more reliable.

The delivery device may also comprise a separate cooling element provided in close proximity to, in particular direct physical contact with, the device operation lock and/or the thermosensitive element for cooling the thermosensitive element in order to relock the delivery device.

In embodiments in which the device operation lock comprises a thermosensitive element as described above, the thermosensitive element may couple the housing of the injection device to a latch, cam, hook, or other retaining means that interacts or engages a movable component such as the cap remover, the cover sleeve, the syringe holder, the dispense spring, and in particular the plunger rod under the eventual or occasional bias of the drive spring. In particular, the thermosensitive element may form a direct, force-fit connection or link between housing and latch, or a form-fit blocking surface preventing a flexibly mounted latch from being driven out of engagement with the movable component. As long as the coupling between housing and latch is unaltered, a driving force of the drive spring intended to urge the movable component in a dispensing direction is compensated, absorbed, counteracted and/or neutralized via the latch. As soon as the coupling has ceased to be operable, that is, as soon as the thermosensitive element is irreversibly destroyed, the latch will move alongside with the movable component, or flex perpendicularly to the direction of movement of the latter, and no longer be able to block the movable component. As only an initial engagement between the latch, cam, or hook and the plunger rod is required, the former may be placed close to a proximal end of the plunger rod, which beneficially results in a short distance from the adjacent thermosensitive element to an electronic control unit likewise located at the proximal end of the injection device. Unlocked axial and/or rotational movement of the plunger rod causes an increased hydrostatic pressure in the liquid medication and results in a skin penetration movement of the syringe and syringe holder instantaneously followed by product dispense.

The order of operation of the device operation lock and the patient-operable trigger element as introduced and denominated above may also be reversed. Trigger element operation, in particular cover sleeve retraction or trigger button pressing, in such case precedes device operation unlocking by way of a device-external signal, and preferably acting directly upon the piston rod and/or the drive spring. Device operation unlocking may well be irreversible in this case, for instance based on a thermosensitive element as detailed above. The unlocking signal in turn may be the response of an injection validation request issued by the injection device upon trigger element operation and detection thereof by a suitable sensing means. In other words, the nominal trigger element acts as a pre-condition on behalf of the subsequent nominal device operation unlock. The injection request may be validated, in an automated manner, by an expert system with access to the relevant device and patient information, such as to minimize any delay until start of the injection by device operation unlock.

Alternative to the above mentioned disposable auto-injector adapted to deliver medication from a pre-filled syringe that is not intended to be replaced or refilled by the patient, other concepts may be employed. In particular, part-reusable, semi-disposable, or hybrid auto-injectors include a disposable unit or reservoir unit comprising the reservoir, and a reusable unit or drive unit comprising the drive means or mechanism. The reservoir unit is discarded once the reservoir is empty, while the drive unit may be used successively for several dose dispensing operations and reservoir units. In such a concept, the reservoir unit includes the device operation lock, in particular the thermosensitive element, whereas the drive unit includes the electronic control unit with the wireless communication unit. Accordingly, a device operation lock in the deactivated and/or unlocked state is replaced by a new, unused instance together with the reservoir unit and the emptied syringe. A straightforward electronic contact between the circuitry of the device operation lock and the electronic control unit enables the latter to control the actuator, in particular the electric heating element, based on control logic and a power source comprised in the drive unit.

According to the invention a method of safely injecting, by way of self-administration, a predetermined quantity of drug from a reservoir through an outlet of a delivery device with a device operation lock preventing a delivery operation of the delivery device as shipped, comprises the steps of:
establishing a communication or contact between the patient and a remote Health Care Professional HCP via telephone or video call, or between the patient and a Health Care Expert System HCES via email or short message text, and inquiring about an intended self-administration;
confirming, or approving, by the HCP or HCES, in particular following an anamneses of the patient by the HCP and/or a check of therapy plan and device data by the HCES, safety and suitability of the intended self-administration, and authorizing the latter by dispatching or transmitting an unlock command message;
receiving, by a wireless receiver of the delivery device, the unlock command, and
unlocking, by an actuator of the delivery device and instantaneously upon receipt of the unlock command, the device operation lock.

The confirmation by the HCP and/or the HCES is part of an integrated therapy management and allows to monitor or supervise the self-administration of a dose of drug by a remotely located qualified person or entity. In particular, the HCP may proceed to an anamnesis of the patient, to ensure that the patient is in a physical condition to receive the medication, without the patient and/or HCP having to travel. The HCES may proceed to an automated check of the specified delivery device, including verification of the intended drug and dose, expiry date, absence of device recall, and correct storage temperature. The patient is ultimately assured that he or she is about to proceed to the correct administration by being able to use the unlocked device, specifically by being able to remove an outlet cover.

In a preferred embodiment, the method comprises the steps of
assigning, or linking, the delivery device as identified by a Unique Device Identifier UDI, or a package of plural identical delivery devices, to the patient, which may occur by the pharmacy or distributor supplying the identified device or devices to the patient, or by the patient himself or herself; and, at a therapy management system, recording or registering such assignment, and
dispatching or transmitting the unlock command, by the therapy management system, to or for a specific delivery device based a confirmation or approval from the HCP or HCES, and based on the recorded assignment of the delivery device or devices to the patient.

In a preferred embodiment, the method comprises the steps of
detecting, by a device-activation sensor of the delivery device, a user-initiated preparation or handling of the delivery device indicative of an intended or forthcoming drug delivery operation of the device,
sending, by a wireless transmitter of the delivery device, an unlock request message to the HCP or HCES, and
contacting, by the HCP or HCES, the patient, and inquiring about the patient condition to confirm or approve safety and suitability of the intended self-administration, by the HCP.

### BRIEF DESCRIPTION OF THE DRAWINGS

The subject-matter of the invention will be explained in more detail in the following text with reference to preferred exemplary embodiments as illustrated in the attached drawings, of which

FIG. 1 depicts a medical injection monitoring and patient support system;

FIG. 2 is a flow chart of an exemplary method of safely unlocking a delivery device;

FIGS. 3a to 3d depict an auto-injector, a locking slider, and an unlocking actuator of a first embodiment;

FIGS. 4a to 4h illustrate three variants of the locking slider—device cap interaction;

FIGS. 5a and 5b depict an auto-injector and a plunger rod of a second embodiment; and

FIGS. 6a and 6b depict a trigger component and a cross-section view of the second embodiment.

For consistency, the same reference numerals are used to denote similar elements illustrated throughout the drawings.

### DETAILED DESCRIPTION OF PREFERRED EMBODIMENTS

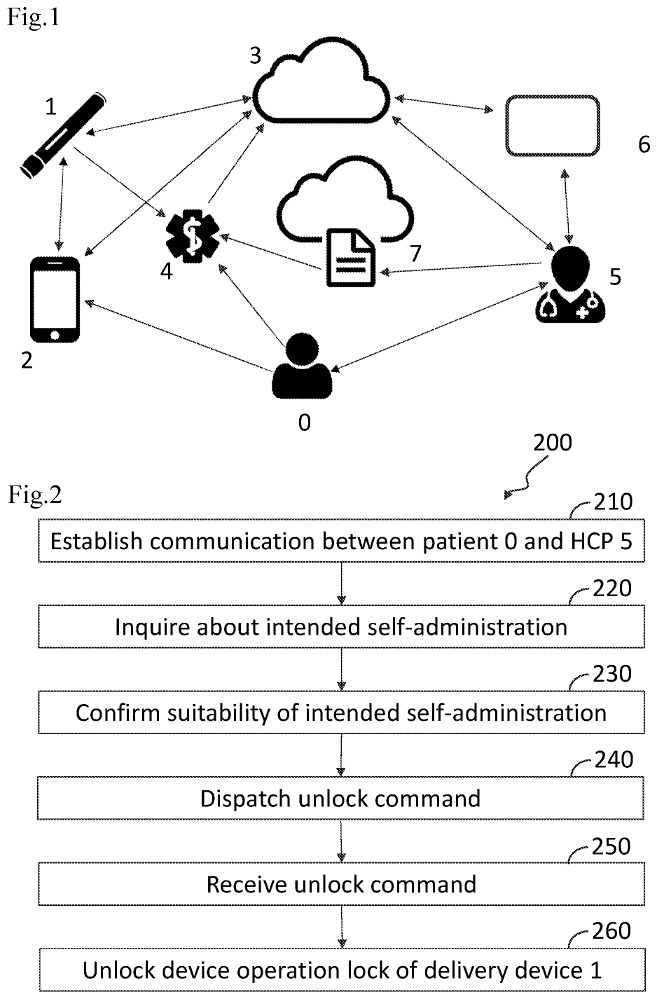

FIG. 1 depicts a medical drug delivery monitoring and patient support system including a delivery device 1 for self-administration of a dose of drug to a patient 0. A smartphone of the patient serves as a user and/or device gateway 2 to a therapy management system 3. A pharmacy 4 is adapted to sell and supply the delivery device 1 to the patient and to provide an association or link between delivery device 1 and patient 0 to the therapy management system 3. A Health Care Professional (HCP) 5 such as a physician is enabled to interact with the patient 0 and the therapy management system 3, optionally via a dedicated tele-health portal or interface 6. An electronic patient file 7 comprising therapy relevant information and/or specific recipes of the patient 0 may be available for reading and writing by the pharmacy 4 and the HCP 5.

The delivery device 1 is a disposable auto-injector with an elongate, pen-shaped device housing essentially symmetric around a main device axis, and a patient-operable trigger element for triggering automated delivery of the dose of drug. The delivery device may also be a disposable patch injector, or wearable bolus injector, with an essentially flat shape factor and with a skin adhesive layer for injection of the dose over prolonged periods of up to 15 minutes. The delivery device may also be a manual device with a delivery lever or button for drug delivery through a force provided by the user.

The delivery device integrates an electronic unit with a short-range communication facility such as Bluetooth Low Energy (BLE) or equivalent short or near range wireless communication technology, such as NFC, WiFi, or IR, to communicate with a nearby gateway device 2 such as a mobile device of the user, including a smartphone or tablet device running a dedicated application program, or a laptop computer configured accordingly. The gateway device 2 in turn communicates with a therapy management system 3 on a cloud-based, distributed data server or computing facility, by way of a 4G/LTE cellular mobile and/or wire-based communication network, and is adapted to relay data between the delivery device 1 or the patient and the therapy management system. Alternatively, the communication facility of the delivery device 1 may communicate directly with the therapy management system 3 via wireless long-range communication networks such as 5G cellular mobile networks, nb-IoT, LTE-m, LoRa, Sigfox, and the patient 0 may interface the data servers of the therapy management system 3 via other communication means. All communication is state-of-the-art encrypted, authenticated and/or otherwise secured in order to comply with relevant regulations applicable to protected health information.

The therapy management system 3 is configured to handle device unlocking requests from the delivery device 1 and unlocking approvals from the HCP 5. To that end, the system receives and registers, in a user account of the patient 0, an assignment or association between the patient 0 and the delivery device 1, either from the pharmacy 2 supplying the device, or from the patient, e.g. by recording and transmitting a Unique Device Identification UDI of the device in the form of printed text, an optical code, or an RFID tag. The UDI includes more generic information about the package in which the specific device was supplied, the production batch, and the drug type, quantity, concentration, and expiry date. The therapy management system may be adapted to store delivery data, drug information, patient adherence data, or any further monitoring data accumulated by adequate sensors of the electronic unit of the delivery device 1 and uploaded to the data server of the therapy management server 3.

The HCP and pharmacy have access to the electronic file of the patient, which comprises at least the prescription for the current therapy, and which may be stored in or next to the user account of the patient on the data server of the therapy management system. The HCP has access to the therapy management system and the registered assignments, or is otherwise given the authority, to have the system dispatch unlock commands based on unlocking approvals from the HCP. The unlock approval of the HCP may be indicating i) any delivery device with the correct drug type, quantity, and concentration as specified, ii) any one device of a production batch as specified, iii) any one device of a package or box of plural devices as specified, or iv) a specific delivery device. The therapy management system is configured to identify, in cases i) to iii), a specific delivery device available to the patient for the intended administration, and to send an unlock command for or to precisely this device.

FIG. 2 is a flow chart of a method 200 summarizing essential steps of an exemplary method of safely unlocking a delivery device with a device operation lock. Method 200 may involve establishing a communication between the patient 0 and the HCP 5 in operation 210; inquiring about intended self-administration in operation 220; confirming suitability of intended self-administration in operation 230; dispatching or transmitting an unlock command in operation 240; receiving an unlock command in operation 250; and unlocking the device operation lock of the delivery device 1 in operation 260.

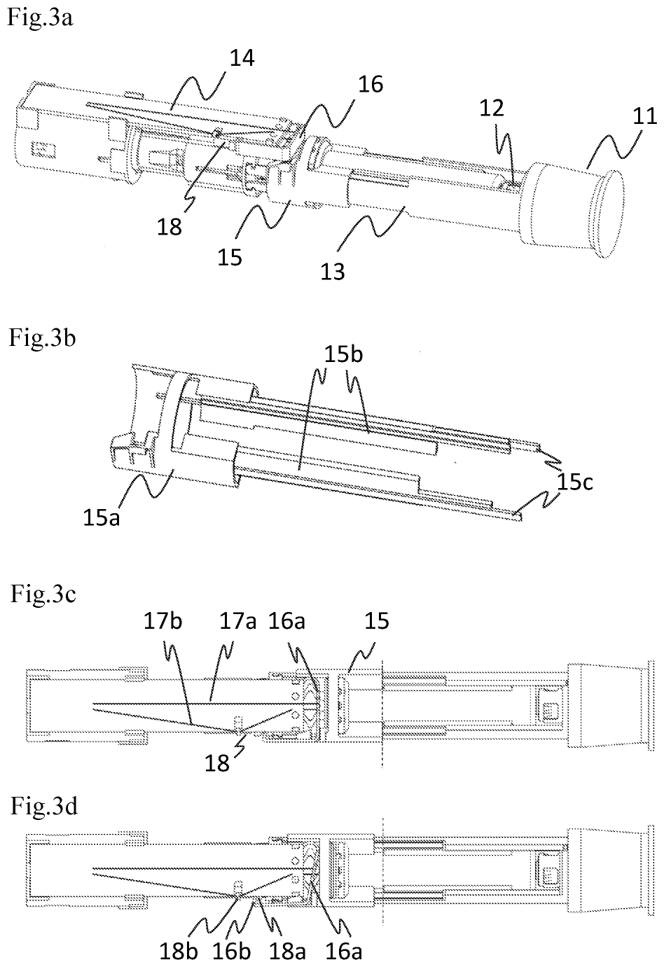

FIG. 3a depicts a perspective view of an auto-injector device 1 according to a first embodiment, in an initial or storage state with a device cap 11 for removing a needle shield of a prefilled syringe 12 being mounted to the device. The housing of the auto-injector has been omitted in order to reveal the axially movable cover sleeve 13 as the trigger element and a drive unit at a proximal end of the device. The latter includes an electronic unit 14 with a Printed Circuit Board PCB mounted immovably in the device and oriented parallel to the main axis of the device. The PCB accommodates sensors for sensing a status of the device and communication means for transmitting and receiving device and/or operation data to or from a nearby mobile device of the patient or a remote expert. The electronic unit 14 also includes an actuator for a cap unlocking mechanism with a shape memory alloy wire arranged on the outer side of the PCB. The actuator is connected to a locking slider 15 that essentially extends from the electronic unit 14 to the device cap 11 and may be shifted axially back and forth a distance corresponding to an actuating hub. A proximal end of the locking slider 15 is axially fixed to a bracket 16 of the actuator and a distal end is adapted and/or arranged to interact with the cap unlocking mechanism. A major part of the locking slider 15 is radially arranged between the cover sleeve 13 and a syringe holder, a proximal flange of which acts as a guiding means for the locking slider 15. The permanent connection between the actuator and the locking slider is established at final assembly, when a syringe unit including the prefilled syringe and the locking slider is coupled to the drive unit including the drive spring and the actuator. The actuator and the locking slider are temporarily axially secured and/or suitably guided by dedicated gripping means in order to enable the connection.

FIG. 3b depicts the locking slider 15 of FIG. 10a in an isolated view. The locking slider comprises a part-cylindrical base 15a at a proximal end and two arms 15b extending therefrom in distal direction. The two arms 15b are radially closer to the center of the device axis and obscured by the cover sleeve 13 in the view of FIG. 3a. A radial blocking surface at the tip or distal end 15c of the arms interacts with the cap unlocking mechanism as detailed below.

FIG. 3c and FIG. 3d depict a top view of the unlocking actuator in the locked state, FIG. 3c, and in the unlocked state, FIG. 3d. A first or unlocking shape memory alloy wire 17a is arranged parallel to the main axis, with a proximal end thereof fixed to a first electrical contact on the PCB, and with a distal end thereof fixed to the metallic actuator bracket 16 as a second electrical contact. Specifically, the distal end is connected to an axially flexible actuator arm 16a of the bracket, which is biased and in turn biases the locking slider 15 in the distal direction by virtue of the elastic properties of the metallic bracket 16. For actuating the cap unlocking mechanism, a current is passed through the first wire 17a, which causes the wire to heat up to a critical temperature at which phase change sets in, and to correspondingly reduce its length by 2-5%. By way of example, a commercial shape memory alloy wire with a diameter of 0.05-0.1 mm and a length of 50-70 mm may develop a hub of 1.5-3 mm within 1-2 seconds when heated to a temperature of 70° or higher by a current of 100-200 mA This contraction in turn pulls the actuator arm 16a and the locking slider 15 in a proximal direction against the bias mentioned. A transverse ledge 16b of the actuator arm 16a engages a proximally oriented blocking surface 18a or edge of a transversally movable anchor 18. Such engagement prevents the locking slider 15 in the unlocked state from shifting distally under the bias of the flexible bracket arm. The actuator includes a second, or locking, shape memory alloy wire 17b connected at both ends to corresponding contacts and forming a kink or deviation angle at an intermediate pulley 18b of the anchor 18. For actuating a cap locking mechanism, or for de-actuating the cap unlocking mechanism, a current is passed through the second wire 17b, which in turn contracts and pulls the intermediate pulley 18b and the anchor 18 in a transverse direction in an attempt to decrease the deviation angle. The transverse ledge 16b passes out of engagement with the blocking surface 18a of the anchor 18, and the actuator arm 16a and locking slider 15 move distally under the distal bias of the elastic bracket 16. Alternatively, the second wire may be arranged to urge the transverse ledge 16b out of engagement with a stationary blocking surface. The blocking surface 18a and the transverse ledge 16b, or at least a counterpart surface thereof, may be made of electrically conductive material and build a contact switch of which the closed state signals the device cap unlocking state.

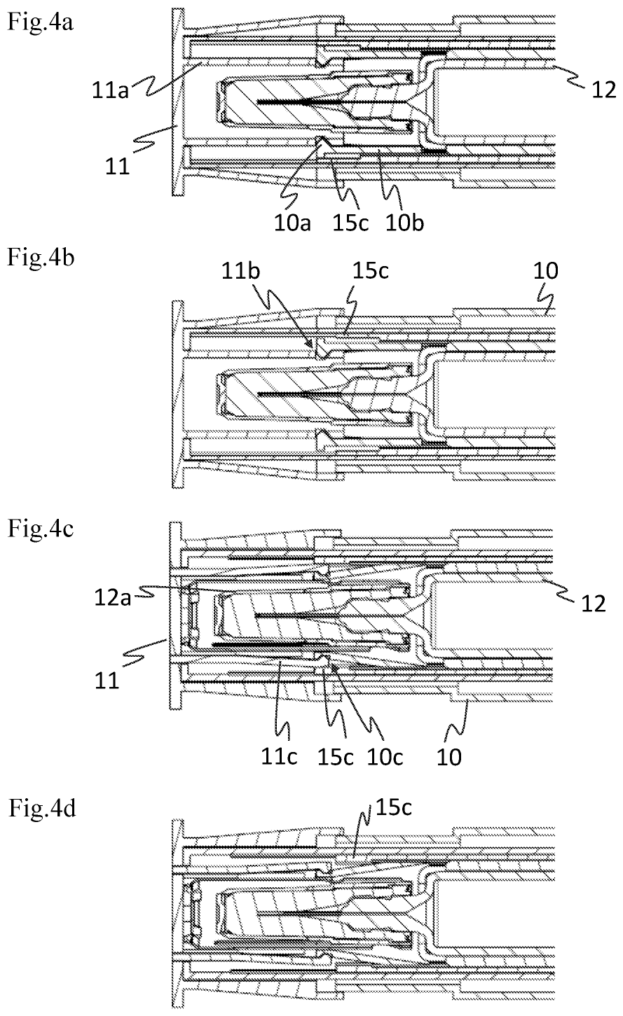

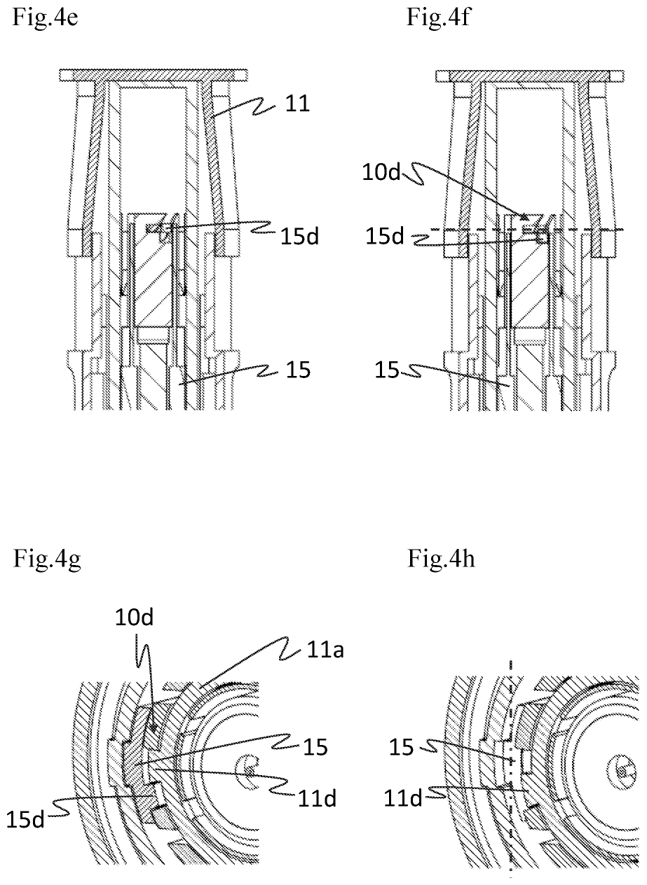

FIG. 4a and FIG. 4b depict a longitudinal section along the main axis of the auto-injector with a first variant of the locking slider—device cap interaction in the locked state (FIG. 4a) and in the unlocked state (FIG. 4b). An inner sleeve 11a of the device cap 11 comprises a recess 11b engaged in the locked state by a locking ledge 10a at a distal end of a flexible locking arm 10b integral with the housing 10. A distal tip 15c of the locking slider 15 prevents the locking ledge 10a from radial movement and from disengaging the recess 11b. In the unlocked state the locking slider 15 has moved proximally by an unlocking hub allowing the locking ledge 10a to flex radially, by virtue of an inclined contact surface at the locking ledge 10a and/or the recess 11b, when the device cap 11 is pulled distally.

FIG. 4c and FIG. 4d depict a longitudinal section along the main axis of the auto-injector with a second variant of the locking slider—device cap interaction in the locked state (FIG. 4c) and in the unlocked state (FIG. 4d). In this variant the radially flexible locking arm 11c is part of the device cap 11, and the locking ledges thereof, in the locked state, engage a recess 10c formed in the housing 10. The locking arm may be part of an inner sleeve of the device cap, or may be part of a metallic remover sleeve of the device cap 11, adapted to engage a rigid needle shield 12a sealing the needle of the pre-filled syringe 12.

FIG. 4e to FIG. 4h depict an off-center longitudinal section along a plane tangential to the inner sleeve of the device cap (FIG. 4e and FIG. 4f) and a cross section perpendicular to the main axis (FIG. 4g and FIG. 4h) of the auto-injector with a third variant of the locking slider—device cap interaction in the locked state (FIG. 4e and FIG. 4g) and in the unlocked state (FIG. 4f and FIG. 4h). In this variant, removal of the device cap 11 includes first a rotation by a few degrees as depicted in FIG. 4g and FIG. 4h, followed by a combined rotational-axial unscrewing movement. The plane of the perpendicular cross section is indicated with a broken line in FIG. 4f, and the intersection of the perpendicular and the longitudinal section plane is depicted by a dash-dot line in FIG. 4h. A rotating cam 11d extending radially outwards from the inner sleeve 11a of the device cap 11 is guided by a cam-path 10d in the housing, wherein a locking cam 15d extending radially inward from the locking slider 15 into the cam path prevents the rotating cam 11d from being rotated in the locked state. In the unlocked state, the locking slider 15 is shifted proximally, and the locking cam 15d is moved out of the blocking engagement with the rotating cam 11d.

Alternative to the shape memory alloy based materials, the actuators may include a magnetic core shifted by the magnetic field of an electromagnet or a solenoid; a DC motor, a stepper motor, or a linear motor, all with or without gearing, Further possibilities include actuators based on a piezo-effect, a bimetal, electroactive polymers, air cushion, or an H2 cell, or a one-time operational melting fuse.

The locking arm or the recess of the previous variants may be part of the cover sleeve instead of the housing. The locking arm may be arranged to flex radially inward upon locking slider retraction and device cap retrieval. The distal head of the locking slider may be positioned, in the locked state, between two off-center flexible locking arms of the device cap engaging two recesses of the housing facing each other. Upon locking slider retraction, these two locking arms may be deflected towards each other in a tangential direction. Alternative variants of the locking slider—device cap interaction include a distally oriented locking arm of the device cap or a distally oriented claw of the remover sleeve abutting a proximally oriented stop surface of the housing, and a locking slider being rotated or shifted distally to urge the locking arm out of engagement with the stop surface. Further concepts involve pivotal levers, or a lock wheel that may be rotated in the unlocked state by less than 180° by the device cap being removed, against a biasing spring.

The electronic unit being integrated in the delivery device may comprise a visual, audible and/or tactile status indicator indicating to a user a status of the system. The status of the system may include a device status of the delivery device or a delivery status of a drug dispensing process. The status indicator may be simple and limited to a few Light Emitting Diodes LEDs in traffic-light colors and/or an audible signal generator for generating language-independent beep sounds or simple melodies. In particular, the status information may include an indication about a lapse of a minimum holding, delay, or dwell time following completion of a substance dispensing activity to inform the user that it is now safe to remove the delivery device. The status indicator may explicitly exclude any advanced human-machine interfacing capability, and be limited to a few, specifically less than ten, messages conveyable to the user. In particular, the delivery device may be devoid of a display, screen, or projector for visually transmitting readable instructions, and likewise exclude an artificial speech assistant for reading out loud the instructions.

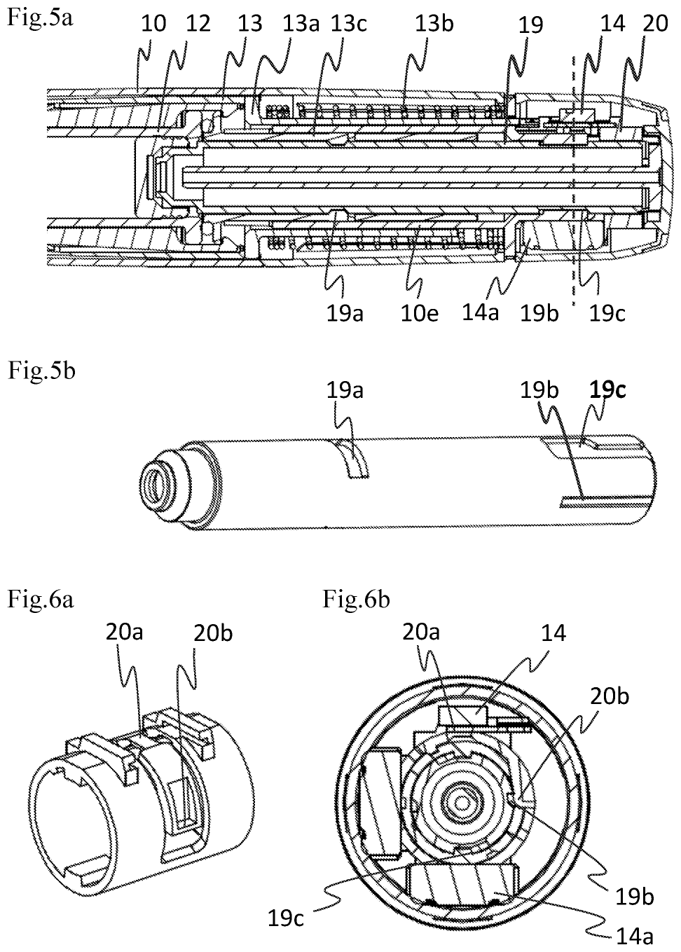

FIG. 5a and FIG. 5b depict a partial axial section of an auto-injector according to a second embodiment of the invention in a locked or shipping state (FIG. 5a), and an isometric view of the plunger rod (FIG. 5b). The auto-injector includes a housing 10 defining a main or longitudinal device axis, a prefilled syringe 12, a cover sleeve 13, an electronic unit 14, a battery 14a, a plunger rod 19, and a trigger component 20 mounted rotationally and axially fix in the housing 10. A lock sleeve 13a is arranged proximally to and in force-fit contact with the cover sleeve 13, and biased by a cover sleeve spring 13b in a distal direction. An injection sleeve 13c in contact with a proximal flange of the prefilled syringe 12 has radially flexible arms with inward-oriented cams kept in engagement with a first recess 19a of the plunger rod by an adjacent blocking surface of a mechanics holder 10e solidly mounted in the housing 10. The plunger rod 19 comprises, in addition to the first recess 19a perpendicular to the main axis, a second, axial recess 19b, and a third recess 19c with a distally oriented guiding surface that is oblique with respect to the main axis, in other words neither parallel nor perpendicular thereto.

A solenoidal compression spring (not shown) is arranged inside the sleeve-shaped plunger rod as a drive or injection spring and exerts an axial force on the plunger rod. The mechanics holder 10e includes a proximally oriented counter guiding surface such that under an unobstructed axial bias of the drive spring, the plunger rod 19 first moves in a screwing movement, including a rotation by a few degrees, before pursuing a pure axial movement. The angular width of the first recess 19a corresponds to said rotational movement, such that the flexible arms of the injection sleeve 13c, and the injection sleeve as a whole, move only axially.

FIG. 6a and FIG. 6b depict an isometric view of the trigger component (FIG. 6a) and a cross section perpendicular to the main axis in a plane denoted by the broken line in FIG. 5a in a shipping state of the auto-injector according to the second embodiment (FIG. 6b). The trigger component includes a device operation lock with a thermosensitive element 20a and a latch 20b. In a locked state, the latch 20b engages the second recess 19b of the piston rod and prevents the latter from rotating by compensating or absorbing the torque generated by the drive spring via the guiding surfaces of recess 19c and mechanics holder 10e. Due to the orientation of the guiding surfaces, the torque acts in clockwise direction when viewed in distal direction, resulting in a compressive strain on the thermosensitive element 20a. The thermosensitive element 20a connects the latch 20b to a body of the trigger component 20 and ultimately to the housing 10. The thermosensitive element thus forms a direct, force-fit connection or link between the housing and the latch. A heating element as the actuator is part of the electronic unit 14 and located adjacent to the thermosensitive element 20a, and may generate sufficient heating power to cause the latter to melt.

In alternative embodiments, an axial instead of a rotational lock, with a latch being connected to the housing in axial direction, may be employed to directly prevent an axial dispense movement of the piston rod or other movable component. Further alternatively, the thermosensitive element may include a form-fit blocking surface preventing a flexibly and even elastically mounted latch from being driven out of engagement with the biased plunger rod. Advantageously, the unconnected or unblocked latch is retained in a position or space where it may no longer interfere with the piston rod or other movable component.

When the cover sleeve as the manually operated trigger element is pushed against the injection site and retracted proximally, a pin or other protrusion at the proximal end of the lock sleeve operates a switch or other trigger element operation detector of the electronic unit. If the unlock command has been received beforehand, the electronic unit instantaneously powers the heating element. Alternatively, an injection validation request is issued by the injection device upon trigger element operation, and receipt of an unlocking approval in response is awaited before the heating element is operated. Heat from the latter is conducted to the thermosensitive element, which results in weakening and destruction of the thermosensitive element, unlocking of the latch, and ultimately a movement of the piston rod in distal direction. The injection sleeve is dragged proximally until the flexible arms are free to flex radially and decouple from the piston rod, which results in tissue penetration of the needle of the pre-filed syringe. The penetration depth corresponds to the distance between the first recess of the plunger rod and the distal end of the adjacent blocking surface of the mechanics holder in the shipping state.

While the invention has been described in detail in the drawings and foregoing description, such description is to be considered illustrative or exemplary and not restrictive. Variations to the disclosed embodiments can be understood and effected by those skilled in the art and practicing the claimed invention, from a study of the drawings, the disclosure, and the appended claims. In the claims, the word “comprising” does not exclude other elements or steps, and the indefinite article “a” or “an” does not exclude a plurality. The mere fact that certain elements or steps are recited in distinct claims shall not preclude the existence of further meaningful combinations of these elements or steps.

LIST OF REFERENCE NUMERALS

| | |
|---|---|
| 1 | injection device |
| 2 | Gateway |
| 3 | therapy management system |
| 4 | Pharmacy |
| 5 | health care professional |
| 6 | health portal |
| 7 | electronic patient file |
| 10 | housing |
| 10a | locking ledge |
| 10b | locking arm |
| 10c | recess |
| 10d | cam path |
| 10e | mechanics holder |
| 11 | device cap |
| 11a | inner sleeve |
| 11b | recess |
| 11c | locking arm |
| 11d | rotating cam |
| 12 | prefilled syringe |
| 12a | needle shield |
| 13 | cover sleeve |
| 13a | Lock sleeve |
| 13b | Cover sleeve spring |
| 13c | Injection sleeve |
| 14 | electronic unit |
| 14a | battery |
| 15 | locking slider |
| 15a | base |
| 15b | arm |
| 15c | tip |
| 15d | locking cam |
| 16 | actuator bracket |
| 16a | actuator arm |
| 16b | transverse ledge |
| 17a, b | SMA wire |
| 18 | anchor |
| 18a | blocking surface |
| 18b | pulley |
| 19 | Plunger rod |
| 19a, b, c | recess |
| 20 | Trigger component |
| 20a | Thermosensitive element |
| 20b | latch |

What is claimed is:

1. A delivery device for self-administration of a predefined amount of drug from an outlet of a reservoir, comprising: a device operation lock preventing a delivery operation of the delivery device as shipped; a wireless receiver for receiving an unlock command; and an actuator for unlocking the device operation lock and configured to be activated instantaneously upon receipt of the unlock command.

2. The delivery device of claim 1, wherein the delivery device is an auto-injector or a patch-injector.

3. The delivery device of claim 2, wherein the device operation lock comprises an outlet cover lock for preventing removal of an outlet cover sealing the outlet of the reservoir.

4. The delivery device of claim 3, wherein the outlet cover lock is adapted to be re-locked by the actuator before the delivery operation has occurred.

5. The delivery device of claim 3, wherein the outlet cover lock is a device cap lock comprising a flexible ledge engaging a counter-ledge and is prevented from disengaging by a locking slider.

6. The delivery device of claim 1, further comprising a wireless transmitter adapted to send an unlock request message.

7. The delivery device of claim 6, further comprising a device-activation sensor to detect a user-initiated preparation of the delivery device.

8. The delivery device of claim 1, further comprising drug delivery sensor means configured to monitor and report the delivery operation of the delivery device.

9. The delivery device of claim 1, further comprising an actuator comprising a shape-memory-alloy element.

10. The delivery device of claim 1, wherein the device operation lock comprises a thermo-sensitive element configured to melt, evaporate, soften and/or otherwise become structurally weakened when subjected to an elevated temperature.

11. The delivery device of claim 10, wherein the actuator comprises heating means for heating the device operation lock and/or the thermo-sensitive element to the elevated temperature conditionally upon the receipt of the unlock command.

12. The delivery device of claim 11, wherein the device operation lock is adapted to engage a proximal end of a plunger rod of an auto-injector prior to the receipt of the unlock command, and wherein a syringe of the auto-injector is adapted to be moved in a distal direction by the plunger rod in an unlocked state.

13. A method of unlocking a delivery device comprising a device operation lock for preventing a delivery operation of the delivery device as shipped, the delivery device for self-administration by a patient of a predetermined quantity of drug from a reservoir through an outlet of the delivery device, the method comprising:
establishing an electronic communication between the patient and a Health Care Professional (HCP) or a Health Care Expert System (HCES), and inquiring about an intended self-administration;
confirming, by the HCP or the HCES, suitability of the intended self-administration by transmitting an unlock command;
receiving, by a wireless receiver of the delivery device, the unlock command; and
activating, instantaneously upon receipt of the unlock command, an actuator of the delivery device to unlock the device operation lock.

14. The method of claim 13, further comprising:
assigning, at a therapy management system, the delivery device to the patient; and
transmitting, by the therapy management system, the unlock command following a confirmation by the HCP and based on the assignment.

15. The method of claim 13, further comprising:
detecting, by a device-activation sensor of the delivery device, a user-initiated preparation of the delivery device;
sending, by a wireless transmitter of the delivery device, an unlock request message to the HCP or HCES; and
contacting, by the HCP or HCES, the patient.

16. The method of claim 13, wherein the delivery device is an auto-injector or a patch-injector, the method further comprising:
activating, instantaneously upon the receipt of the unlock command, the actuator of the delivery device to unlock an outlet cover sealing the outlet.

17. The method of claim 16, further comprising:
re-locking the outlet cover by the actuator before the delivery operation has occurred.
